(19) United States
(12) Patent Application Publication
(10) Pub. No.: US 2009/0071104 A1
(43) Pub. Date: Mar. 19, 2009

(54) METHOD AND DEVICE FOR THE STERILE FILLING WITH FLUIDS

(75) Inventor: Sven Fischer, Obertraubling (DE)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: KRONES AG, Neutraubling (DE)

(21) Appl. No.: 11/886,398

(22) PCT Filed: Mar. 10, 2006
(86) PCT No.: PCT/EP2006/002209

§ 371 Date: Oct. 21, 2008

(30) Foreign Application Priority Data
Mar. 16, 2005 (DE) 10 2005 012 507.7

(51) Int. Cl. B65B 55/10; A61L 2/20; B67C 7/00

(52) U.S. Cl. 53/426; 53/561; 53/268; 53/471

(57) ABSTRACT

A device for the sterile filling of liquids in bottles, having a sterilizer which is used to sterilize the bottles with H₂O₂, a filling element which is used to fill the bottles, a closing element which is used to apply a closing element as a closing lid, and structure to adjust the temperature of the bottle whereby condensation of the H₂O₂ on the surface of the bottle is prevented. Also, a method for sterile filling of liquids in bottles comprising the steps of the bottles are sterilized with H₂O₂, then filled and closed. During sterilization, the bottles have such a temperature that condensation of the H₂O₂ is prevented on the surface of the bottles.

### CROSS-REFERENCE TO RELATED APPLICATION

The present application claims the benefit of priority of International Patent Application No. PCT/EP2006/002209 filed on Mar. 10, 2006, which application claims priority of Germany Patent Application No. 10 2005 012 507.7, filed Mar. 16, 2006. The entire text of the priority application is incorporated herein by reference in its entirety.

The disclosure relates to a method and a device for the sterile filling with fluids, such as used in beverage bottling and packaging container operations.

### BACKGROUND OF THE DISCLOSURE

Sterile filling with beverages, for example, is important to achieve a long minimum shelf life. For this purpose, it is known to sterilize bottles before the filling, to fill them with sterile beverages, and then close the bottles while maintaining the sterility.

From DE 37 01 079 A1, for example, a method is known for the sterilization of packaging containers. Here, hydrogen peroxide is blown onto the preheated container, so that a condensate film forms on the walls of the container. Then the sterilizing agent is removed by rinsing with sterile water. The drawback here is that a large quantity of wastewater is produced by rinsing with sterile water.

From DE 196 42 987 A1 a method is known for sterilizing, filling and closing packaging containers. It is disclosed here to preheat packaging containers with hot air and then introduce a sterilization agent, such as, for example, hydrogen peroxide. The sterilization agent here undergoes some condensation. After the sterilization, the containers are dried to reliably remove any residues of the sterilization agent. The drawback here is that high energy costs are generated for the separate drying to remove the sterilization agent.

From DE 32 35 476 A1 a method is known for sterilizing packaging material, in which a hydrogen peroxide-containing sterilization agent is atomized, then blown, in a mixture with air, onto the surface of the packaging material to be sterilized, and caused to condense on it. Furthermore, it is known from the above to heat the surface to be sterilized, before blowing the steam-air mixture onto it, to a temperature which is equal to the dew point temperature of the steam-air mixture, or slightly lower. The condensate produced has to be removed again by later blowing air on or into the packaging material.

### SUMMARY OF DISCLOSURE

The problem of the disclosure is to produce a device and a method for sterile filling, which needs as little energy and/or produces as little wastewater as possible, while allowing as rapid as possible sterilization.

The device comprises a sterilizer for sterilizing bottles with H₂O₂. Furthermore, a device for filling and a device for closing the bottles are provided. Due to the closing, the filled product of the bottle is protected hermetically against contamination, and can thus be released into a nonsterile environment.

Furthermore, means are provided for adjusting the temperature of the bottles so that H₂O₂ does not condense on the bottles. While, in the state of the art, condensation of H₂O₂ is provided to ensure a good sterilization, the H₂O₂ is applied to the bottles here at a temperature so that the H₂O₂ does not condense, but remains gaseous instead. This procedure as well produces a sufficient sterilization effect. However, the need to rinse, or to dry the bottle with excessive energy consumption is avoided.

Using a temperature above the dew point, it is also possible to choose a sufficiently high temperature to allow a very rapid sterilization due to the thermally increased reaction rates.

With the device it is possible, for example, to sterilize PET bottles, which are usually manufactured with an injection molding machine, particularly a stretch blow molding machine, from preforms. The injection molding machine can here be connected with the sterilizer by a conveyor or a transfer device. However, the bottles can also be manufactured independently of the sterilizer.

It is advantageous to adjust the temperature of the bottles by means of an appropriate coolant in the blow molding machine. In injection molding, the preforms are preheated, so that the material of the preform becomes deformable. The bottles manufactured in this manner are hot after the injection molding process and they are cooled in the injection molding machine, for example, by blowing in cooling air, or by using water-cooled molds. In the process, the bottles are usually cooled to temperatures of 20-30° C. If the cooling is less intense, the temperature of the delivered bottles can also be elevated, so that the bottles are released from the injection molding machine at a temperature of at least 50° C., for example, 50-60° C. At a corresponding H₂O₂ pressure or H₂O₂ partial pressure, this is sufficient to prevent a condensation of H₂O₂ in or on the bottles. The required temperature of the bottles also depends on the temperature of the H₂O₂. The lower the temperature of the H₂O₂ is, the higher the temperature of the bottles must be, because cold H₂O₂ can lower the surface temperature of the bottles by a slight cooling action.

It may be advantageous not to arrange the blow molding machine or the sterilizer immediately next to each other, to maintain accessibility to both installations. However, an intermediate conveyor must be intercalated between the blow molding machine and the sterilizer. This conveyor can be a simple sliding rail, along or down which the suspended bottles slide, suspended by their neck, or a conveyor with a conveyor belt, an air conveyor, a conveyor with grippers, or similar apparatus.

The path that the bottles must cover between the blower and the sterilizer is here advantageously insulated thermally. This can be carried out by a simple sheathing of the path, for example, with Plexiglas panes, glass, refined steel or a similar material, which merely ensures that the warm air which flows out of the blow molding machine, or is generated by the warm bottles, is kept in the area of the bottles. The sheathing prevents heat loss by convection. In addition, heat insulating materials can also be used to further improve the thermal insulation. The heat loss of the bottles can be decreased by the sheathing or the insulation, so that they are still at a sufficiently high temperature after a longer transport duration, to prevent H₂O₂ condensation.

On the path that the bottles cover as they move to the sterilizer, a heating device can also be provided. It allows warm bottles for the sterilization to be made available independently of an injection molding machine.

To warm the bottles, a nozzle can be provided, by means of which warm air is blown into the bottles, to warm the bottles from inside. The warm air is generated preferably with hot steam, because this is a rapid and cost effective procedure. In the process, the steam can be, for example, superheated, and mixed with air. The air can also be heated in a heat exchanger. The air preferably has a temperature of 100-150° C. The higher the temperature is, the more rapidly the warming of the bottles can occur. However, if the temperatures are excessively high, a PET bottle can undergo deformation, so that the work can be carried out at an air temperature of approximately 70° C. in case of particularly sensitive bottles.

An external heater can also be provided for warming; it causes hot air to flow against the bottles from the exterior. The temperature of the air is preferably 50-60° C. Higher outside air temperatures require more energy and lead to strongly increased heat losses, because the external air is in contact with colder machine parts. However, since PET is a plastic which does not present good heat conductivity, it is advantageous to warm the bottles not only from inside, but also from outside, because doing so achieves shorter warming durations. However, it is also possible to warm from the exterior only.

To minimize the heat losses, a tunnel can be provided, in which the heating device can heat the bottles.

It is preferred to provide a nozzle or a gas outlet by means of which H₂O₂ can be blown in or allowed to enter the bottles in a gas stream or in an H₂O₂-air mixture. With nozzles H₂O₂ can be blown into the bottle cavity so is well distributed in it, and all the areas of the bottle interior are properly gassed. The nozzle or the gas outlet is preferably immersible in the bottle to achieve a better concentration of H₂O₂ in the bottle.

The nozzle or the gas outlet can be preceded by a connected air heater, which preheats the air for the nozzle or for the gas outlet. An H₂O₂ injection nozzle can be provided upstream or downstream of the air heater. In this way, a preheated H₂O₂-air mixture can be generated, by means of which the sterilization can be carried out well and rapidly.

To blow the H₂O₂ out again, it is also advantageous to provide a nozzle or a gas outlet by means of which a sterile gas, such as, for example, sterile air can be introduced into the bottles. Here, it is preferred to provide a prewarming device for prewarming the sterile gas to prevent the condensation of H₂O₂ as a result of cold air being blown in. If the bottle temperature is sufficiently high, however, H₂O₂ also does not condense on the bottles if sterile air that has not been prewarmed is blown in.

It is preferred to provide means by means of which the external side of the bottles can be sterilized. This can be achieved, for example, with a sterilization tunnel, which keeps H₂O₂ that flows out of the bottle on the external side of the bottles. In this way, any H₂O₂ that has been lost on the bottle interior can be used for the external sterilization, and thus the H₂O₂ consumption can be reduced. To ensure an appropriate H₂O₂ concentration outside of the bottles, additional H₂O₂-air mixture connections can be provided. To reach an appropriate temperature in the sterilization tunnel, both hot and cold air connections can be provided. At the appropriate temperature, H₂O₂ does not condense in or on the bottles.

Upstream of the sterilizer, a heat insulating tunnel can be provided, by means of which the warmed bottles can be maintained at the desired temperature. As a result, one can ensure that the bottles do not undergo cooling on the way from the heating device or an injection molding machine to the sterilizer, and stay instead at the desired temperature. This section can also be used for an external treatment with an H₂O₂-containing atmosphere. As a result, the external sterilization is further improved.

Advantageously, the bottle closures are also sterilized with H₂O₂. To prevent the condensation of H₂O₂ on the bottle closures, a prewarming device is provided, by means of which the bottle closures can be heated to a temperature above the dew point of H₂O₂. This can be done by applying hot air, or by infrared irradiation. Furthermore, it is advantageous for the bottle closures to be sterilized in a reservoir, because this maximizes the duration of exposure to H₂O₂.

Furthermore, it is advantageous to provide means which thermally insulate the entire sterilizer. This can be achieved with a sheathing with simple partitions made of refined steel, Plexiglas, glass or a similar material. However, one can also use other thermally insulating materials. The results of the sheathing is that the warmed air remains with the sterilizer. By warming the entire sterilizer to a temperature above the dew point of H₂O₂, one prevents the condensation of H₂O₂ at other places. Such condensation in itself would not be detrimental to the sterilization of the bottle, but it would lead to increased H₂O₂ consumption, and maybe to liquid H₂O₂ runoff, which is not desirable.

In addition, a heater can also be provided by means of which the entire sterilizer can be heated. This can be achieved with a hot air blower, electrical heating elements, or other devices. In this way a complete and sufficiently even warming of the sterilizer can be achieved easily.

Furthermore, other bottle or closure treatment apparatuses can be insulated thermally by sheathing and/or heat insulation, or they can be heated additionally. Such bottles or closure treatment apparatuses can be, for example, a bottle reservoir, preferably a dynamic bottle reservoir. As a result, one can achieve the effect that the bottles do not undergo excessive cooling on the path between the injection molding machine or heating device and the sterilizer.

To heat the different devices, it is possible to use, for example, hot air, which is produced in the blow molding machine or during the heating of the preforms. This hot air can be used, for example, to heat a bottle reservoir, the sterilizer, the path between the blow molding machine and the sterilizer, or similar devices. Optionally, additional heaters can be provided, if the hot air which is produced during the injection molding process or the preheating of the preforms is insufficient. This may be particularly relevant when the production rate of the machine is increased.

In the method according to the disclosure, bottles are sterilized with H₂O₂, and then sterile filled and closed. During the sterilization, the bottles have a temperature which prevents the condensation of the H₂O₂ in or on the bottles. As a result of the injection molding process, the bottles preferably have the required temperature. However, they can be heated optionally to the required temperature with hot air, or by infrared irradiation, or with other heating devices. Furthermore, it is advantageous to convey the bottles after the injection molding process, with thermal insulation, i.e., in a state where they are jacketed or enclosed with heat insulating materials, to prevent in this way a strong cooling of the bottles.

Advantageously, the sterilization is carried out either by blowing in H₂O₂, or by blowing it out, for example, with sterile air. This can lead to a rapid and sufficiently strong sterilization. Because the removal of the H₂O₂ can be carried out in a fairly simple way, and thus requires little time, the duration of exposure to H₂O₂ can be increased without an increase in the overall residence time of the bottles in the sterilizer in comparison to the state of the art, because less time is needed to remove the H₂O₂ in comparison to conventional methods.

In the method, the sterilizer is preferably heated in its entirety, or partially, to a temperature such that the parts that come in contact with H₂O₂ are at a temperature above the dew point of H₂O₂.

It is particularly advantageous to use methods where, between the injection molding machine and the sterilizer, a reservoir for bottles is provided, which is preferably a dynamic reservoir, working on the FI-FO (first in-first out) principle. With it, the injection molding process can be continued in case of failure of the sterilizer/filler, and the manufactured bottles can be temporarily stored in the reservoir. For this purpose, the intermediate reservoir is preferably designed so that the bottles in the reservoir do not undergo any cooling, or do not undergo a strong cooling, rather they are maintained at a temperature, or continue to be actively warmed. As a result, stored bottles can be sterilized as well without condensation of H₂O₂. Moreover, maintaining the temperature of the bottles as they come from the blow molding process prevents the need for additional energy to heat the bottles, which leads to an energy saving.

The air used at the different places, such as, for example, the air that is being enriched with H₂O₂, the air to be blown out, or the air for heating, is preferably ionized air. The ionized air is manufactured in an ionizer at high voltage, and it facilitates the removal of particles.

### BRIEF DESCRIPTION OF THE DRAWINGS

Several embodiments of the devices and of the method are explained in reference to the drawing. In the drawing:

FIG. 1 is a schematic representation of a device,

FIG. 2 is a schematic representation of a device with a reservoir, and

FIG. 3 is a schematic representation of a device with its own heating device.

### DETAILED DESCRIPTION OF THE DISCLOSURE

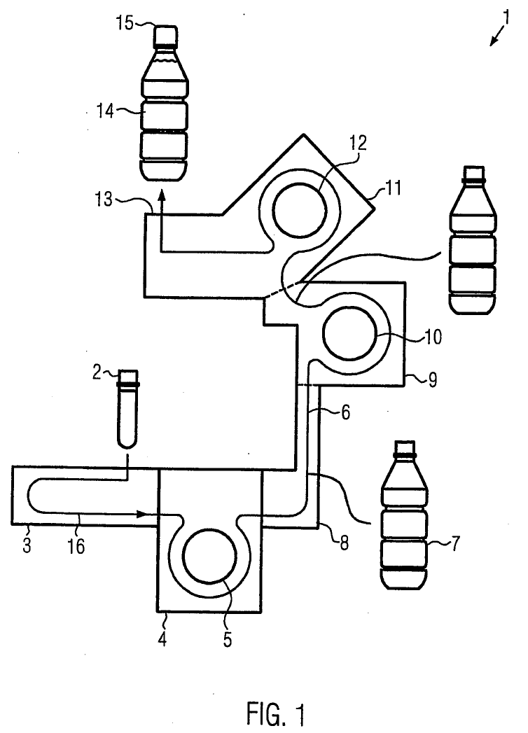

FIG. 1 shows a device 1 with a blower machine 4 in the form of a stretch blowing machine, a sterilizer 9, and a filler and closing device 11. The blower 4 can have a carousel 5, around which the blow molds circulate. In front of the blower 4, a heating station 3 with conveyor chain 16 is connected, in which the preforms 2 made of PET are warmed to the processing temperature.

The cooling of the blow molds is regulated in such a way that the finished bottles 7 leave the blower 4 at a temperature of approximately 50-60° C.

Downstream of the blower 4, a path section 6 between the injection blower 4 and the sterilizer 9 is represented. Finished shaped bottles 7 are transported along the path section 6. To achieve this, a corresponding appropriate conveyor (conveyor with grippers, slide rail, air conveyor, etc.) can be provided. The conveyor can, for example, use the neck at the upper end of the bottles 7 below the bottle threading to hold the bottles.

The path section 6 is arranged in a tunnel 8, which encloses the path section 6. The tunnel 8 can be constructed, for example, from refined steel, Plexiglas, glass or a steel glass construction or a similar material. It is also possible to surround the tunnel 8 with a thermal insulation material.

The path section 6 is followed by a sterilizer 9. The latter can also have a carousel 10, which is provided with corresponding feed and delivery stars. Along the path that the bottles take around the carousel, or on the periphery of the carousel, nozzles are provided to blow H₂O₂ into the bottles 7. Furthermore, nozzles are provided with which sterile air can be blown into the bottles 7, to blow out the H₂O₂. The nozzles for blowing in H₂O₂, as well as the nozzles for blowing in air, can be the same nozzles, which are, in that case, connected to two corresponding feed lines, for H₂O₂ and for sterile air.

The H₂O₂ is generated advantageously by evaporating H₂O₂ on a hot plate in a separate installation (for example, a flash evaporator). For this purpose, it can be applied by metering in liquid form to a hot plate, for example, by dripping. The gaseous H₂O₂ produced in this way is then directed to the nozzle or the nozzles through corresponding pipes.

The H₂O₂ gas can be used either in pure form, or mixed with another gas, such as, air, nitrogen, oxygen, steam, or a similar substance.

It is preferred to use an H₂O₂-hot air mixture. At the time of the release, the mixture has a temperature of 150° C., for example. As a result of the high temperature, the sterilization effect is good. However, lower temperatures are also possible (energy saving). Even higher temperatures may lead to bottle deformations, depending on the volume stream and the treatment duration.

The nozzles or the gas outlets for the mixture are arranged on a carousel. Here, normal environmental air is filtered and optionally dried, and then directed via a rotating distributor to the rotating part of the carousel 10. There, the H₂O₂ generated in a flash evaporator is introduced through a nozzle into the air path. Upstream or downstream of the injection nozzle, the air or the air-H₂O₂ mixture is heated in an electrical heater. If the heater is arranged downstream of the injection nozzle, the air is heated, for example, to 200-300° C., preferably 250-300° C. For several nozzles or gas outlets, a common flash evaporator can be provided. In the bottle, the temperature can be accordingly lower.

The hot mixture is directed to the bottles 7 for a predetermined duration, such as, for example, for 2 seconds to 10 seconds, preferably for approximately 8 seconds. The H₂O₂ does not condense in the bottles. During the treatment duration, a reduction rate of up to log 6 can be reached.

At the times when the nozzle or gas outlet does not introduce a mixture into bottles, the mixture produced is removed via a bypass to the bottle exterior treatment. This occurs, for example, when no bottle 7 is held at the nozzle.

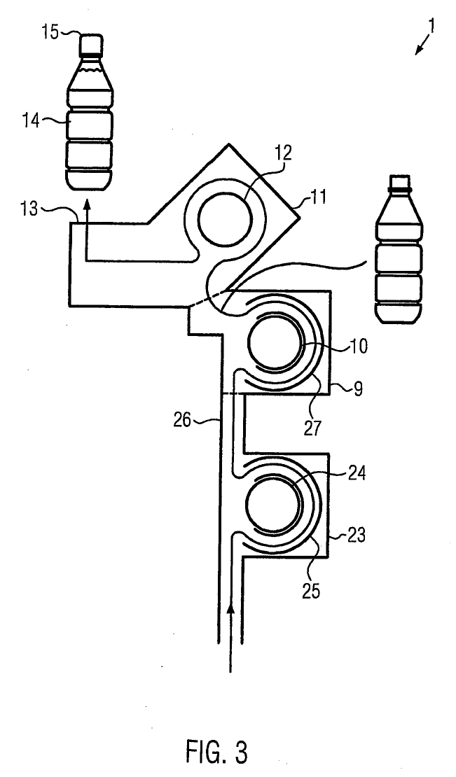

For the bottle exterior treatment, the path of the bottles 7 around the carousel 10 is enclosed in a tunnel (see reference numeral 27 in FIG. 3). As a result, a sterilizing atmosphere can be generated in the area of the bottle path. The temperature in the tunnel (hereafter referred to as the sterilization tunnel to distinguish it from other tunnels) can be adjusted to approximately 50° C. by the introduction of hot or cold air. At this temperature, H₂O₂ does not condense. During the operation, a small amount of the H₂O₂-hot air mixture flows out of the bottle interior, out of the bottles, thus enriching the atmosphere in the sterilization tunnel with H₂O₂. In addition, H₂O₂ can be introduced into the tunnel (in the form of a gas, or a mixture). This H₂O₂ can come from the bypass of the bottle interior treatment, or it can be supplied through a separate feed line for H₂O₂, and/or hot and/or cold H₂O₂-air mixture.

To minimize losses of H₂O₂ out of the sterilization tunnel, the tunnel is as gas-proof as possible. It is preferred to provide ground seals between the movable and the fixed parts of the sterilization tunnel.

Regardless of whether a sterilization tunnel is provided or not, the sterilizer 9 can be jacketed. The purpose of this is, on the one hand, to prevent H₂O₂ gas from entering into the environment. On the other hand, the warm air should be kept at the sterilizer 9. The sheathing of the sterilizer 9 can be achieved by an appropriate covering or a similar process. It is best for the covering to be gas-proof. It is also possible to apply a slight low pressure in the area of the sterilizer 9, to prevent H₂O₂ from flowing out through possible leaks in the sheathing, and instead suck in normal air. In addition, the escape of H₂O₂ gas is prevented.

Along the way that the bottles 7 take moving into the sterilizer 9 and out of it, small openings, which exactly fit the bottles 7, can be provided, for example (drawn with broken lines in the figures), for the purpose of allowing bottle transport, while ensuring the sheathing of the sterilizer 9 or of the sterilization tunnel. Locks can also be provided, to remove and reintroduce the bottles 7 from and into the sterilizer 9.

Downstream of the sterilizer 9, a filler and closing device 11 is arranged. It also presents a carousel 12, on whose periphery filling valves are provided for filling the bottles 7 with filling product 14 in the form of a sterilized beverage. Furthermore, the bottles 7 can be closed with closure caps 15. The filler and closing device 11 is also sheathed, to ensure sterile conditions during the filling and the closing. The bottles 7 that have been closed in this way can be delivered out of the device 1 at the outlet 13. The closing of the bottles 7 can occur either on the filler carousel 12, or on a closing device arranged downstream of the filler.

For the sterilization of the bottle closures 15, they are kept in a reservoir at a temperature of 45-60° C., where they are gassed with H₂O₂. Between the reservoir and the closing device, a transport device for the bottle closures is provided. On this path between the reservoir and the closing device, blow nozzles are arranged, to allow the removal of H₂O₂ from the closures. Furthermore, the temperature of the bottle closures can be lowered with the blower itself, or with another cooling device downstream of the closing device, to ensure the dimensional stability of the bottle closures.

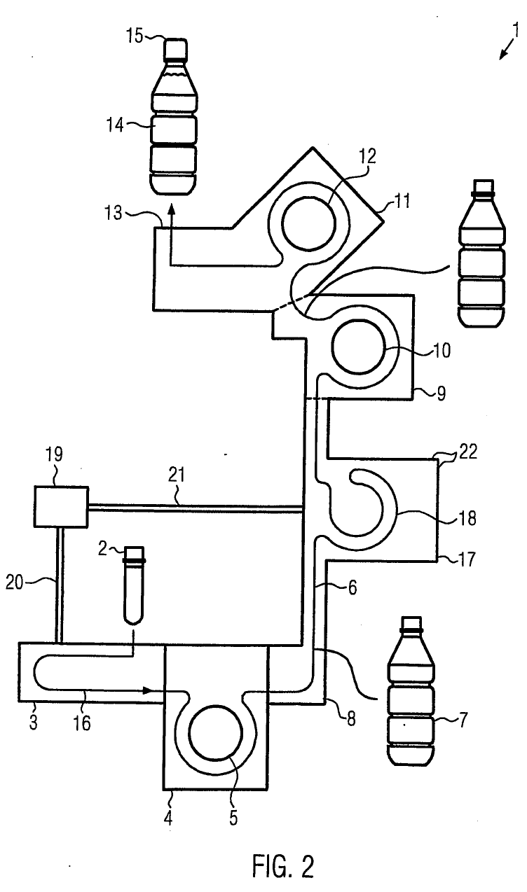

In FIG. 2, the device from FIG. 1 is additionally arranged with a reservoir 17 between the blower 4 and the sterilizer 9. This reservoir 17 can compensate for brief capacity differences between the blower 4 and the sterilizer 9, or filler 11. For example, when the sterilization is stopped, or the filling is stopped, the blower 4 can continue to produce bottles that are taken up in the reservoir 17. Their number can then be reduced subsequently by acceleration of the sterilization/filling and/or of the slowing of the blower 3. If the blower 4 stops, the reservoir 17 can still be emptied completely or partially, to conduct the sterilization and the filling continuously until the blower 4 can resupply bottles 7.

The reservoir 17 comprises advantageously twisting tracks 18, along which the bottles are transported, where the length of the twisting track 18, through which the bottles 7 in the reservoir 17 pass, is variable. The result is a dynamic bottle reservoir 17.

The bottle reservoir 17 is encapsulated with a wall 22, to hold the warm air, which exits from the form blower 4, or which is generated by the heat of the bottles 7, at the bottles 7. The wall 22 can be constructed, for example, from metal, plastic, such as, Plexiglas, glass, or a similar material, to encapsulate the warm air in the case of the twisting tracks 18. The walls 22 of the reservoir 17 can also comprise heat insulating material.

The cavity of the reservoir 17 can also be heatable with warm air. For this purpose, normal air can be warmed, or the waste air from the blow molding process can be used. In the heating device 3, for example, by heating the preforms 2, warm air is produced, which can be directed through a pipe 20 to a control or regulation unit 19, which blows warm air, in a controlled way, through a pipe 21 into the reservoir 17. The reservoir 17 then must have an appropriate air outlet. The line 21 can also end in the area of the path section 6. In the unit 19, or upstream/downstream of the latter, in the pipes 20, 21, an additional heating device can also be provided, for the purpose of heating air if no, or an insufficient amount of, warm air is generated in the prewarming device 3. The pipe 20 can also branch off away from the blower 4, or it can comprise an additional feed line from the blower 4 to the control and regulation device 19.

The sheathing or covering of the different, in each case adjacent, devices, such as, of the path 6, of the reservoir 17, of the sterilizer 9, and of the filler 11, can also be provided jointly. In FIG. 2, for example, the left covering or sheathing of the path 6, of the reservoir 17, and of the sterilizer 9, is represented as a cohesive unit.

FIG. 3 shows an additional embodiment, in which the bottles 7 are heated with a heating device 23 to a desired temperature. The heating device 23 comprises a carousel 24 on whose periphery the bottles 7 can circulate. On the periphery of the carousel 24, nozzles are arranged, by means of which hot air can be blown into the bottles. The air has a temperature of approximately 100-150° C. To heat the air, a steam feed is provided, by means of which the hot steam can be mixed with the air, to prewarm the air in this way. A heat exchanger or another air heater can also be provided to heat the air. The humidity of the air is such that no condensate forms in the bottles. The air that has not been heated and the steam are preferably directed to the rotating part via, in each case, a separate rotary distributor. The heating of the air occurs first in the rotating part of the carousel. As a result, the air can be heated only immediately before the release, so that it cannot undergo cooling on the way to the delivery, which would lead to energy losses.

The hot air line also has a bypass, which directs the hot air past the nozzles into the tunnel 25. The purpose of the tunnel 25 is to hold the hot air in the path of the bottles 7 to achieve an external warming. To adjust the temperature in the tunnel 25, one or more warm or cold air connections can be provided additionally. To generate the hot air, heating cartridges, for example, are provided, which are located a short distance before the air inlet in the tunnel 25. The heater is designed in such a way that an air temperature of 50-60° C. is reached in the tunnel. This corresponds to the temperature at which the bottles are to be heated.

In FIGS. 1-3, downstream of the sterilizer 9, a path section is shown, which the bottles must move through to reach the sterilizer (see, for example, path 6 in FIGS. 1 and 2, and path 26 in FIG. 3). This section can be predetermined by a conveyor, or by one or more transfer stars. The section is arranged preferably in a tunnel 8, 26 (hereafter called the heat insulating tunnel to distinguish it). Warm air, which contains H₂O₂, can enter, for example, from the sterilizer 9 into the heat insulating tunnel 8, 26. In addition, connections for and/or hot and/or cold H₂O₂-air mixture can be provided on the heat insulating tunnel 8, 26. It is preferred to generate an air stream in the heat insulating tunnel which is opposite the direction of movement of the bottles (countercurrent procedure). For this purpose, a suction device can be provided at the bottle inlet of the heat insulating tunnel, which sucks the H₂O₂-containing air out of the sterilizer through the heat insulating tunnel, and then removes it. The heat insulating tunnel 8, 26 is preferably connected with seal to the sterilization tunnel 27, so that no H₂O₂ or hot air can escape at the transition. In addition, the connection between the tunnel 25 and the heat insulating tunnel 26 is preferably gas-proof.

The method is explained in reference to FIGS. 1-3. Preforms 2 are introduced into heating station 3, in which they are warmed. The processing temperature of the preforms is higher than 120° C. The path of the preforms 2, or the path of the bottles 7 with the conveyor chain is represented schematically with the line 16. The warmed preforms 2 are transferred into the blower 4, where they are blown to bottles 7. After the stretch blow molding process, the bottles 7 present an elevated temperature, which is partially the result of the warming of the preforms in the heating station 3. Usually, the bottles are cooled to as low a temperature as possible. In the present method, the bottles, however, are not cooled to the lowest possible temperature, rather they are left at a temperature of 50-70, preferably 50-60, ° C.

The bottles 7 are delivered into the tunnel 8 at such a temperature that, after passing through the tunnel, they still have a sufficiently high temperature. To this effect, the tunnel 8 is designed in such a way that the bottles 7 lose little or no heat, to maintain a sufficiently high temperature.

As shown in FIG. 3, the bottles 7 can also be heated with a heating device 23 to the required temperature. For this purpose, hot air is blown into the bottles 7. The air is here heated with steam, heat exchanger, an electrical heater, or a similar device, to a temperature of 100-150° C. The hot air introduction lasts approximately 3-7 sec, preferably approximately 5 sec.

Moreover, the bottles 7 are heated from the exterior. For this purpose, the bottles 7 are transported in a tunnel 25, in which the air temperature is approximately 40-60° C. The temperature in the tunnel 25 is the result of, on the one hand, the hot air introduction into the bottles 7, and also of hot air which reaches the tunnel 25 through the bypass past the hot air nozzles. At times when no hot air is to be introduced into the bottles, the hot air flows through the bypass into the tunnel 25. As a result, the hot air generation can be operated continuously, while the bottles 7 are exposed directly to hot air for only a defined duration.

To maintain a temperature of 50-60° C. in the tunnel 25, hot air is blown additionally into the tunnel 25 using heating cartridges. For this purpose, a temperature control is provided. Then, the bottles 7 are transferred into the tunnel 26 (heat insulating tunnel). In the latter, the temperature is 50-60° C., so that the bottles 7 do not undergo cooling, rather they are maintained at the desired temperature. Furthermore, the tunnel holds an H₂O₂-containing atmosphere, so that the bottles 7 are pre-sterilized, at least from the exterior. After the transport through the heat insulating tunnel 26, the bottles 7 are guided past the sterilizer 9.

The bottles 7 are introduced at a sufficiently high temperature into the sterilizer 9, so that H₂O₂ does not condense during the gassing with H₂O₂ in the sterilizer 9. For this purpose, on the one hand, the hot H₂O₂-air mixture (temperature 125-175° C., preferably 150° C.) is injected with a nozzle into the bottles, and the bottles 7 are exposed on their exterior side to an H₂O₂-containing atmosphere. The external atmosphere has a temperature of approximately 50-60° C. Because the condensation is prevented, the gaseous H₂O₂ can consequently be injected simply by blowing sterile air into it, and the bottles that have been sterilized in this way are filled and closed, and then delivered.

For closing, the bottle closures 15 are sterilized. For this purpose, the closures are kept at a temperature of approximately 50-60° C. in a reservoir, and exposed there to H₂O₂. The residence time can range from several seconds to several minutes. The closures are then transported out of the reservoir, and in the process the H₂O₂ is removed from the closures by being blown or sucked off. The closures are also cooled in the process, so that they are dimensionally stable and can be handled easily. The closures that have been sterilized and cooled in this way are guided under sterile conditions to the closing device.

After the delivery of the bottles 7 from the molding blower 4 or from the heating device 23, the bottles can also be introduced into a reservoir 17, in which they are transported along twisting tracks, to achieve a storage effect in this way. The length of the twisting track is here variable, to achieve a variable buffer size. The reservoir 17 is designed so that the bottles 7 lose little heat during storage, or rather so their temperature is maintained. For this purpose, for example, warm air from the prewarming device 3 is transferred through a feed line 20 to a regulation and control unit 19, which then guides the air along a duct 21 into the inner cavity of the reservoir 17. The control and regulation unit 19 in the process establishes a temperature in the interior of the reservoir 17 which is sufficiently high so that H₂O₂ does not condense on the bottles 7 during the sterilization in the sterilizer 9.

What is claimed:

1. Device for the sterile filling with fluids of bottles, comprising:
a sterilizer for sterilizing the bottles with H₂O₂,
a filler for filling the bottles,
a closing device for the application of a closure, and means for adjusting the temperature of the bottles (7) can be adjusted so that a condensation of H₂O₂ on the bottle surface is prevented.

2. Device according to claim 1, wherein cooling agents in a blow molding machine for manufacture of the bottles are used to adjust the temperature of the bottles (7) at an outlet of the blow molding machine (4).

3. Device according to claim 2, wherein the means comprises one of sheathing, thermal insulation or a combination thereof, of the path (6) of the bottles (7) between the blow molding machine (4) and the sterilizer (9).

4. Device according to claim 1, wherein the means comprises a heating device (23) for the path of the bottles (7) to the sterilizer (9).

5. Device according to claim 4, wherein the heating device (23) comprises an internal heating device.

6. Device according to claim 5, wherein to prewarm the air, a steam feed is provided, by which the air can be heated to a temperature above the desired temperature of the bottles (7).

7. Device according to claim 4, wherein the heating device (23) comprises an external heating device, by which the bottles (7) can be heated from the exterior.

8. Device according to claim 4, wherein the heating device (23) comprises a tunnel (25) around the bottle path.

9. Device according to claim 1, and one of at least one nozzle or at least one gas outlet, is provided for introducing H₂O₂ in gaseous form into the bottles (7).

10. Device according to claim 9, wherein an air heater is provided to generate hot air for the nozzle.

11. Device according to claim 10, wherein an injection nozzle device for injecting H₂O₂ through a nozzle into the path of the air is provided.

12. Device according to claim 1, wherein the sterilizer (9) comprises a sterilization tunnel (27) for sterilizing the bottles (7) with H₂O₂.

13. Device according to claim 12, and feed lines are provided for one of the H₂O₂, the hot and/or cold H₂O₂-air mixture, and the hot and/or cold air to the sterilization tunnel (27), or a combination thereof.

14. Device according to claim 1, and wherein the bottle path is surrounded, immediately upstream of the sterilizer (9), by a heat insulating tunnel (8, 26), in which the bottles (7) can be maintained at the desired temperature.

15. Device according to claim 14, wherein at the transition between the heat insulating tunnel (8, 26) and the sterilizer (9), feed lines to the heat insulating tunnel (8, 26) are provided, for one of H₂O₂, hot and/or cold H₂O₂-air mixture, or a combination thereof.

16. Device according to claim 1, and wherein a nozzle or a gas outlet is provided, by which sterile gas, can be introduced into the bottles (7) to blow out the H₂O₂.

17. Device according to claim 1, and wherein a pre-warming device is provided for bottle closures (15), by which the bottle closures (15) are heated.

18. Device according to claim 17, and an application device is provided, by which a flow of H₂O₂ can be applied to the bottle closures (15) in such a way that the H₂O₂ does not condense.

19. Device according to claim 17, and a reservoir for bottle closures is provided, in which the bottle closures (15) are maintained at a temperature such that H₂O₂ present in the reservoir does not condense.

20. Device according to claim 1, wherein the means for adjusting the temperature comprises one of sheathing, thermal insulation, heating of the sterilizer (9), or a combination thereof.

21. Device according to claim 1, wherein the means for adjusting the temperature comprises one of a sheathing (22), a thermal insulation a heating of additional bottles or closure treatment devices, or a combination thereof.

22. Method for the sterile filling with fluids of bottles, comprising the steps:
sterilizing the bottles with H₂O₂,
filling as well as closing the bottles, and
during the sterilization the bottles (7) present a temperature such that condensation of H₂O₂ on the bottle surface is prevented.

23. Method according to claim 22, wherein the bottles (7) have the required temperature due to the injection molding.

24. Method according to claim 23, and conveying the bottles (7), after the injection molding process, with thermal insulation to prevent excessive heat loss of the bottles (7).

25. Method according to claim 22, and heating the bottles (7) to the required temperature.

26. Method according to claim 25, wherein the air is heated with steam.

27. Method according to claim 25, wherein the heating of the bottles occurs during the circulation around a carousel (24).

28. Method according to claim 22, and blowing the H₂O₂ into the bottles (7) as one of a pure gas, gas-air mixture, gas-air-steam mixture, or combination thereof, and then blowing out again with air.

29. Method according to claim 28, wherein to blow in H₂O₂, one of a nozzle or a gas outlet is immersed in the bottle (7).

30. Method according to claim 28, and injecting the H₂O₂ through a nozzle into an air path, where the injection through a nozzle can occur one of upstream or downstream of an air heater.

31. Method according to claim 22, and bringing the bottles (7) in contact from the exterior with H₂O₂ for the sterilization.

32. Method according to claim 31, wherein the H₂O₂ for the external contact one of flows out of the bottle interior and is guided to the bottles (7) from the exterior.

33. Method according to one of claim 22, and transporting the bottles (7), after the warming, to a sterilizer in a heat insulating tunnel (8, 26) with a warm atmosphere, to maintain the bottles (7) at the required temperature.

34. Method according to claim 33, and one of transferring the warm from the sterilizer (9) into the heat insulating tunnel (8, 26), and guiding one of H₂O₂ hot and/or cold H₂O₂-air mixture, and/or hot and cold air to the heat insulating tunnel (8, 26).

35. Method according to claim 22, and pre-warming the bottle closures (15) and one of at the same time and subsequently exposing the bottle closures (15) to a flow of H₂O₂ for sterilization.

36. Method according to claim 35, and exposing the bottle closures (15) to one of a flow of H₂O₂ and a flow in a reservoir for bottle closures (15).

37. Method according to claim 22 and one of heating and maintaining the sterilizer (9) at an elevated temperature, so that a condensation of H₂O₂ on parts of the sterilizer (9) is prevented.

38. Method according to claim 22, and storing the bottles (7) in a warm state between injection molding and sterilizing.

39. Method according to claim 22, and wherein the bottles (7), during the sterilization, present a temperature of 50-70° C.

40. Method according to claim 22, wherein the air used is ionized.
